(19) United States
(12) Patent Application Publication
(10) Pub. No.: US 2021/0109094 A1
(43) Pub. Date: Apr. 15, 2021

(54) DETECTION OF DISEASE COMPONENTS USING MAGNETIC PARTICLES AND MICROFLUIDICS

(71) Applicant: Case Western Reserve University, Cleveland, OH (US)

(72) Inventors: Robert Deissler, Cleveland, OH (US); Brian Grimberg, Cleveland, OH (US); Umut Gurkan, Cleveland, OH (US); Susann Brady-Kalnay, Cleveland, OH (US); Robert Brown, Cleveland, OH (US); Yuncheng Man, Cleveland, OH (US); Ran An, Cleveland, OH (US)

(21) Appl. No.: 17/068,152

(22) Filed: Oct. 12, 2020

Related U.S. Application Data
(60) Provisional application No. 62/913,878, filed on Oct. 11, 2019.

(51) Int. Cl. G01N 33/543; B01L 3/00; G01N 21/64

(52) U.S. Cl. CPC G01N 33/54333; B01L 3/502715; B01L 3/502761; G01N 21/6428; G01N 2021/6439; B01L 2400/043; B01L 2200/0652; B01L 2200/0642; B01L 2300/0663

(57) ABSTRACT

Disease components can be detected in a fluid using magnetic particles and microfluidics. Magnetic particles are combined with a sample in a fluid. The sample may include disease components. The magnetic particles can be configured to tag any disease components within the sample. The fluid can be forced into a microchamber with two microcompartments. In the first microcompartment, the fluid can be exposed to a magnetic field and/or magnetic field gradient. The tagged disease component can be trapped by the magnetic field and/or magnetic field gradient due to the magnetic particles, allowing nonmagnetic components of the fluid to be washed away while the tagged disease components remain trapped by the magnetic field and/or magnetic field gradient. Then, the disease components can be forced into a more narrow second microcompartment and detected using optical instruments.

### CROSS-REFERENCE TO RELATED APPLICATIONS

This application claims the benefit of U.S. Provisional Application No. 62/913,878, filed Oct. 11, 2019, entitled “LOW-LEVEL DETECTION OF DISEASE IN BODY FLUIDS USING MAGNETIC PARTICLES, MAGNETS, AND MICROFLUIDICS”. This provisional application is hereby incorporated by reference in its entirety for all purposes.

### TECHNICAL FIELD

This disclosure relates generally to detection of disease components using magnetic particles and microfluidics.

### BACKGROUND

Traditionally, disease components (e.g., pathogens or pathogenic materials) can be detected in a patient's body by detecting antibodies generated by the body's immune response. This detection is often delayed from the original infection because a detectable number of antibodies is only generated by the body's immune response some time period after the initial infection. This time delay gives the disease components time to infect the patient's body and cause an associated malady, which may make treatment more difficult. For example, the current standard test for Lyme disease—the Centers for Disease Control and Prevention (CDC) 2-Step test—requires waiting several weeks post exposure before the test is conducted. This delay allows the Lyme disease infection to advance to a level where treatment requires a 14 to 21-day course of oral antibiotics. However, if central nervous system or cardiac symptoms are present, a 14 to 28-day course of intravenous antibiotics is required. These treatments may cause side effects, including a lower white blood cell count, mild to severe diarrhea, or colonization or infection with other antibiotic-resistant organisms unrelated to Lyme disease. Some patients may develop post-treatment Lyme disease syndrome (PTLDS), also known as “chronic Lyme”, where further antibiotics are ineffective.

Early detection without a delay would allow for a more immediate treatment with a lower risk of complications. However, early detection requires detection of the actual pathogen rather than detection of antibodies generated in response to the disease components. In any attempt to detect disease components themselves, it is important to be able to detect very low concentrations of disease components in a fluid. As an example, the concentration of Borrelia bacteria that causes Lyme disease may be as low as a few bacteria/ml of blood in the early stages of the disease. In another example, circulating tumor cells (CTCs), which are indicative of the presence of cancer, can also have very low concentrations in blood (e.g., one cell per mL).

### SUMMARY

The present disclosure relates to early detection of disease components (e.g., pathogens or pathogenic components), allowing for a more immediate treatment with a lower risk of complications. Described herein are devices, systems and methods that employ optical detection of disease components in a fluid using a microchamber and magnetic particles for trapping and detection of the disease components. The trapping and detection can be done in a single device more quickly than traditional analysis techniques.

In accordance with an aspect of this disclosure, a method is provided for performing optical detection of disease components in a fluid using a microchamber and magnetic particles for trapping and detection of the disease components. Magnetic particles can be combined with a sample in a fluid. The magnetic particles can be configured to tag disease components within the sample. The fluid can be forced into a microchamber that is exposed to a magnetic field gradient. The tagged disease component is trapped by the magnetic field gradient. Nonmagnetic components of the fluid can be washed away while the tagged disease components remain trapped by the magnetic field gradient and the disease components can be detected within the microchamber using optical instruments.

In accordance with another aspect of this disclosure, a device (also referred to as a diagnostic device) is provided that can perform optical detection of disease components in a fluid using a microchamber and magnetic particles for trapping and detection of the disease components. The diagnostic device includes a microchamber that includes a first microcompartment with a first cross-sectional area and a second microcompartment with a second cross-sectional area. The second cross-sectional area is less than the first cross-sectional area. The first microcompartment is configured to receive a fluid comprising magnetic particles configured to tag disease components within the fluid. At least one magnet can establish a magnetic field gradient within the first microcompartment of the microchamber. The tagged disease components become trapped by the magnetic field gradient so that nonmagnetic components are washed away. A detector can detect disease components within the second microcompartment of the microchamber using optical instruments as the disease components flow through the second microcompartment of the microchamber.

### BRIEF DESCRIPTION OF THE DRAWINGS

The features, objects, and advantages of the invention will become more apparent from the detailed description set forth below when taken in conjunction with the drawings, wherein:

FIG. 1 is a block diagram of an example diagnostic device that can be used to perform optical detection of disease components in a fluid using a microchamber and magnetic particles for trapping and detection of the disease components;

FIG. 2 is an illustration of an example of the microchamber of FIG. 1 with a first microcompartment for trapping and a second microcompartment for detection;

FIG. 3 is an illustration of example magnet(s) that can be used to establish a magnetic field gradient to trap the magnetic particles;

FIG. 4 is a simplified illustration of a sample with magnetic particles caught in a magnetic field gradient in the trapping area of FIG. 1;

FIG. 5 is a simplified illustration of different example scenarios, one with a disease component and the other without the disease component;

FIG. 6 is an illustration of example of a detection area of the device of FIG. 1;

FIGS. 7-9 are illustrations of example detection schemes used with the second microcompartment of FIG. 6;

FIG. 10 is a process flow diagram of an example method for performing optical detection of disease components in a fluid using magnetic particles and a microchamber for trapping and detection of the disease components;

FIGS. 11 and 12 are process flow diagrams of example methods for detecting disease components;

FIG. 13 shows a microfluidic device with three channels that can be used to execute the method of FIG. 10;

FIG. 14 shows an image of Lyme bacteria (B. burgdorferi) with attached magnetic particles;

FIG. 15 shows images of two samples that were prepared, a without Lyme bacteria and b with Lyme bacteria (B. burgdorferi), and the flow direction in the channels, with the unattached magnetic particles in a and the Lyme bacteria with the attached magnetic particles trapped by magnets in b; and

FIG. 16 shows an image of bound Lyme bacteria (B. burgdorferi) being trapped by magnets.

### DETAILED DESCRIPTION

The present disclosure uses a combination of magnetic particles, magnets, and microfluidics to trap and detect disease components. Notably, the present disclosure can perform the detection in the same device where the trapping is done. This makes the detection quick and easy compared to traditional techniques.

As used herein, the term disease component can refer to at least a portion of any pathogen or pathogenic material that can cause or be indicative of a malady (e.g., a disease, a condition, or the like). For example, disease components can include a bacterium, a virus, a fungus, a parasite, a cell expressing a disease marker, a cell from a tissue biopsy, a cancer cell, or the like. The disease component may be part of a sample, but the sample need not include the disease component.

The disease component can be included within a fluid. The fluid can include a biological fluid originating from inside the body of a living organism, like blood, sputum, urine, sweat, breast milk, synovial fluid, cerebral spinal fluid, blister fluid, cyst fluid, etc., and/or a non-biological fluid, like a buffer. For example, the fluid can include a concentration of the disease component, but may not include the disease component. The sample can be within a fluid or placed within a fluid (e.g., the sample can be cells or tissue from a biopsy). In some instances, at least a portion of the sample and/or the fluid can undergo processes, such as digestion and/or dilution.

More specifically, the present disclosure relates to devices, systems and methods that employ optical detection of disease components in a fluid using a microchamber and magnetic particles for trapping and detection of the disease components. As previously noted, the detection of the disease components can lead to early detection of the disease components (e.g., before patient antibodies detectable by current standard tests are generated) that can allow for a more immediate treatment with a lower risk of complication. Magnetic particles (e.g., to be combined with a binding component, free to be combined with the disease component, or the like) can be added to a sample in a fluid. The magnetic particles can be configured to tag disease components within the sample (e.g., the binding component, also referred to as a bindable agent, can be specific for binding to a certain disease component, such as antibodies, peptides, proteins, etc.).

The fluid can be forced into a microchamber that is exposed to a magnetic field and/or a magnetic field gradient. The tagged disease component and/or free magnetic particles can be trapped by the magnetic field and/or magnetic field gradient. Other non-magnetic components of the fluid are not trapped by the magnetic field and/or magnetic field gradient and can be washed away while the tagged disease components remain trapped by the magnetic field and/or magnetic field gradient. The disease components that were trapped and not washed away can be detected within the microchamber using optical instruments. The detection can be used in cases with a low-level quantity of disease components; however, detection is not limited to low-level detection. The diagnostic devices, systems, and methods described herein can be automated, efficient, and low cost, with the detection able to be completed within a quicker timeframe than traditional detection.

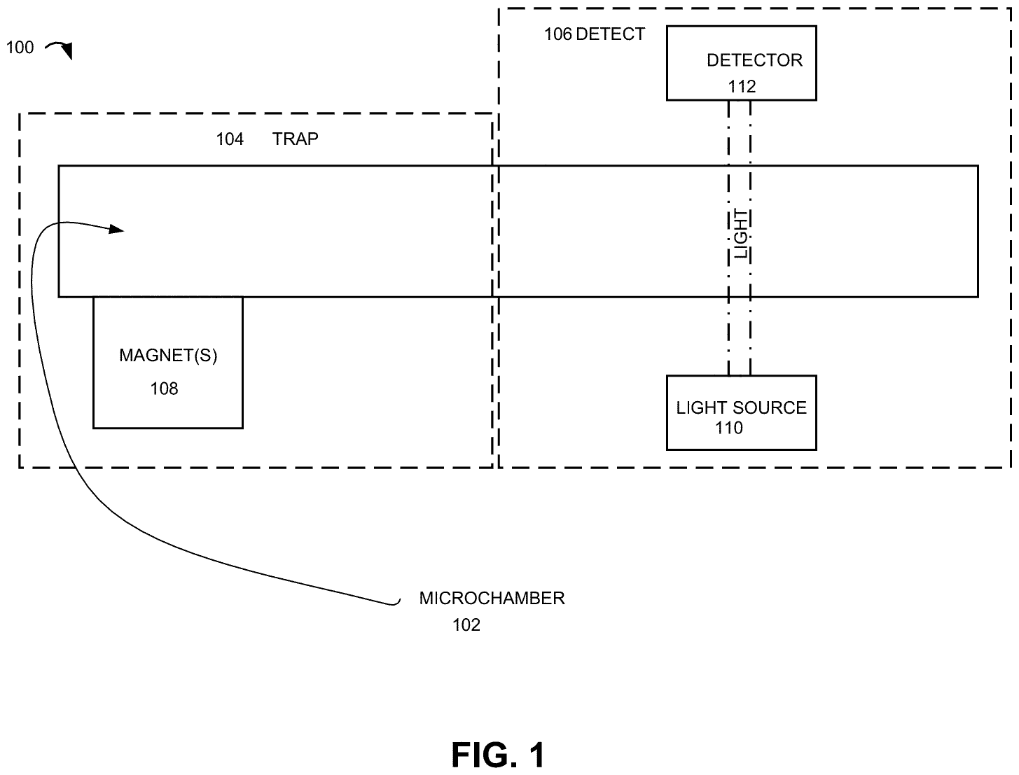

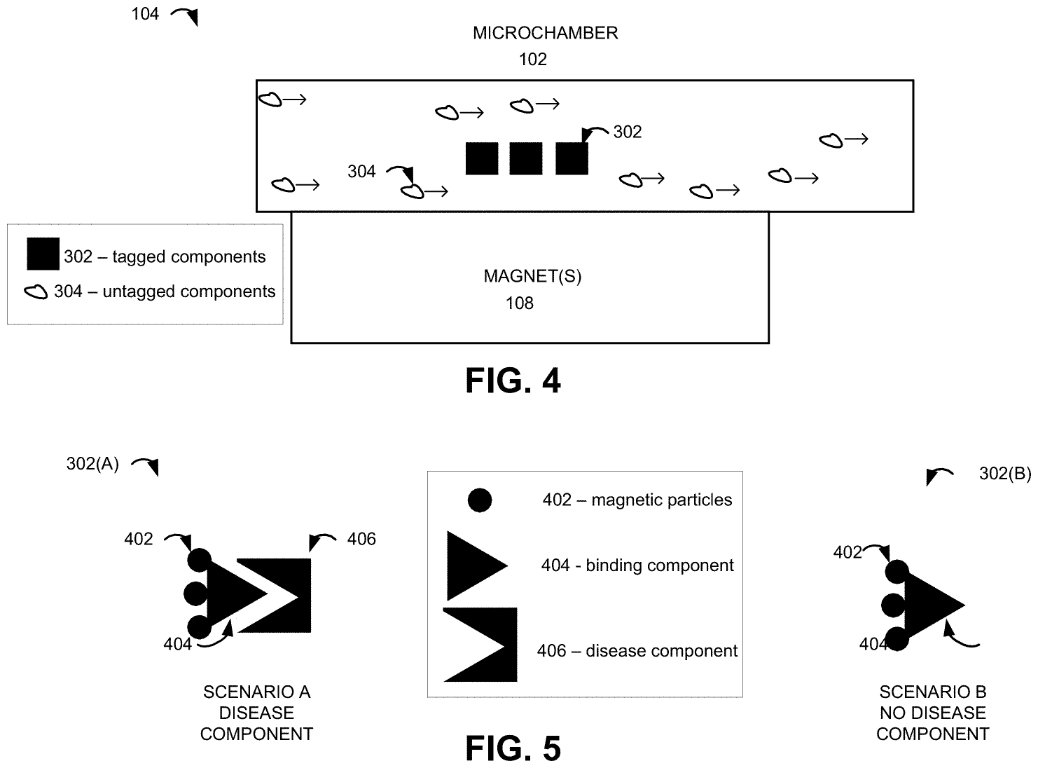

FIG. 1 shows an example device 100 (also referred to as a diagnostic device) that can be used to perform optical detection of disease components in a fluid. The device 100 uses a microchamber 102 for trapping (trapping region 104) and detection (detection region 106) of the disease components. It should be noted that the term “trapping” (and all variations and tenses thereof) should be understood to mean using a “magnetic filter” in a microfluidics flow through to hold back the magnetic components in a fluid while allowing the removal of non-magnetic components in the fluid. In some instances, not all of the magnetic components may be held back—depending on factors, such as the size of the magnetic particles, the number of particles attached to the disease component, the flow rate, or the like, for example. The non-magnetic components can be separated from the magnetic components by using the magnetic filter. For example, a magnetic field and/or a magnetic field gradient can be used to separate out the majority of the magnetic components in the fluid from the non-magnetic components in the fluid, while holding the magnetic components in place (shown, for example, in FIGS. 4 and 5). It should be noted that if the magnetic particles were much smaller than the disease components, for example, a portion of the unattached particles may get removed through the filter, while still trapping any disease component.

The trapping and/or detection can be aided by employing magnetic particles. The magnetic particles can include or be made of any magnetic material that is natural and/or man-made. Each of the magnetic particles can be of any 2-dimensional (e.g., negligible depth) or 3-dimensional shape having a size less than 100 microns (the size can be a distance from one side of the particle to another in a line, like a diameter if the microparticle are of a circular shape). For example, each magnetic particle can have a size less than 50 microns, less than 25 microns, less than 1 micron, etc. As another example, each magnetic particle can have a size greater than 10 nanometers. The magnetic particles, in some instances, can be functionalized to bind, attach to, or otherwise complex with a binding component (which can be specific for binding to a certain disease component, like antibodies, peptides, proteins, etc.) or the disease component itself, so that the magnetic particles can be put into a fluid that may include the disease component, and then bind or otherwise form complexes with any disease component in the fluid.

The device 100 includes a microchamber 102 that includes a trapping region 104 and a detection region 106. The microchamber 102 can be made of glass and/or plastic and may be optically clear and have a width, depth, and length. The surface of at least a portion of at least one interior surface of the microchamber 102 can be functionalized (e.g., with columns, pillars, channels, or the like, on the micro-scale or smaller). The functionalized surface can prevent the disease component from sticking to the surface, for example.

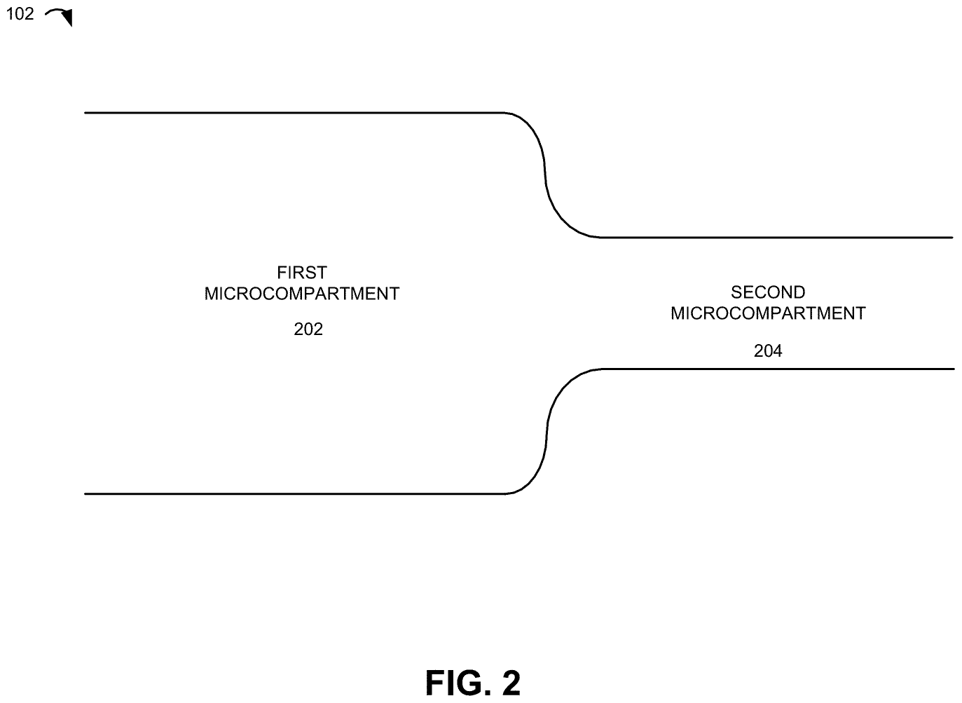

The trapping region 104 and the detection region 106 can each be made of the same material. While the trapping region 104 and the detection region 106 can be in the same compartment, in some instances, the microchamber 102 can include one or more compartments each made of the same material and formed from the same microchamber, but may be differently sized (e.g., a first microcompartment with a first cross-sectional area can be used for the trapping region 104 and a second microcompartment with a second cross-sectional area, which can be less than the first cross-sectional area, and may also be referred to as more narrow than the first region, can be used for the detection region 106). For example, FIG. 2 shows an example where the microchamber 102 includes a first microcompartment 202 and a second microcompartment 204, the second microcompartment 204 being much more narrow than the first microcompartment 202 (e.g., the first microcompartment 202 has a much greater cross sectional area than the second microcompartment 204).

In some instances, the one or more microcompartments can have varying widths, depths, and/or lengths. As an example, the microchamber 102 can have a length of 40 cm or less, a width of 10 cm or less, and a depth of 5 mm or less. As another example, the microchamber 102 be a microfluidic channel having a length of 10 cm or less, a width of 1 cm or less, and a depth between 0.01 mm and 0.5 mm. The portion of the microchamber 102 that includes the trapping region 104 and the portion of the microchamber 102 that includes the detection region 106 can have different parameters (e.g., different depths) such that the trapping region 104 has a larger cross-sectional area than the detection region 106.

At least one magnet (magnet(s) 108) can be within the trapping region 104 (outside the microchamber 102 but next to and/or adjacent to the microchamber 102). The magnet(s) 108 can establish a magnetic field (or magnetic field gradient) within the microchamber 102 (e.g., the first microcompartment 202). As an example, the magnet(s) 108 can include at least two magnets to establish a magnetic field (or magnetic field gradient). As another example, the magnet(s) 108 can include at least four magnets to establish the magnetic field (or magnetic field gradient). It should be noted that the magnet(s) 108 can be moveable into different orientations relative to each other.

The magnet(s) 108 can include one or more simple, inexpensive lab magnets. However, one or more of the magnet(s) 108 can be one or more permanent magnets. Generally, permanent magnets can produce a high magnetic field with a low mass. For example, the magnetic field can be between about 0.01 T and about 100 T. As another example, the magnetic field can be between about 0.1 T and 10 T. As a further example, the magnetic field can be between 0.1 T and 2 T. Additionally, a permanent magnet is generally stable against demagnetizing influences. For example, this stability may be due to the internal structure of the magnet. The permanent magnet can be made from a material that is magnetized and creates its own persistent magnetic field. The permanent magnet can be made of a hard ferromagnetic material, such as alnico or ferrite. However, the permanent magnet can also be made of a rare earth material, such as samarium, neodymium, or respective alloys.

As another example, one or more of the magnet(s) 108 can be an electromagnet. An electromagnet can be made from a coil of a wire that acts as a magnet when an electric current passes through it, but stops being a magnet when the current stops. The coil can be wrapped around a core of a soft ferromagnetic material, such as steel, which greatly enhances the magnetic field produced by the coil.

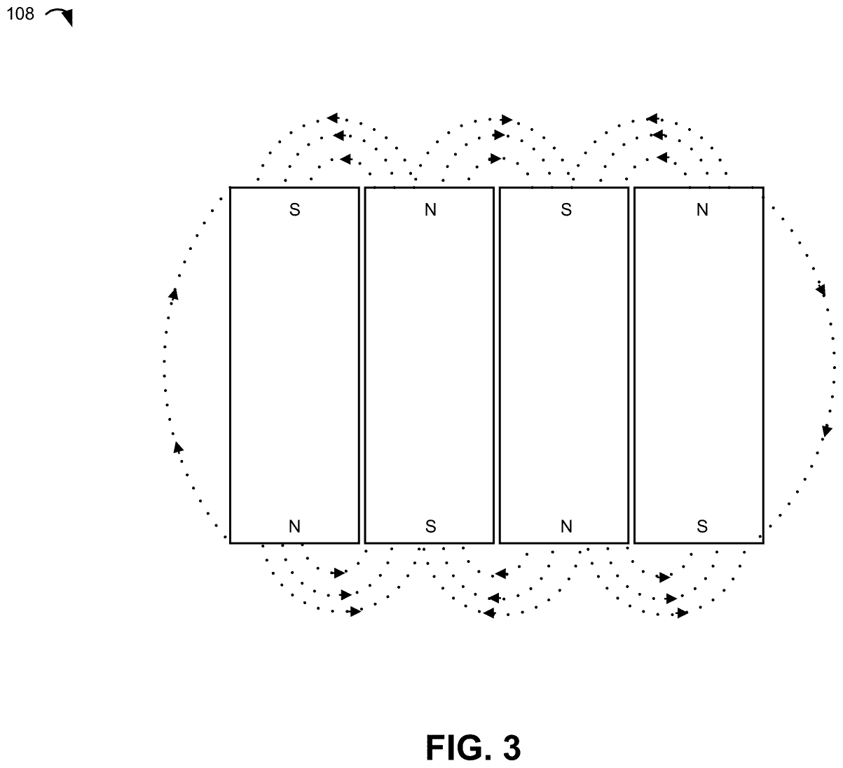

The portion of the microchamber 102 inside the trapping region 104 (the first microcompartment 202 in FIG. 2) can be configured to receive a fluid that includes magnetic particles, either bound to binding components or with binding components delivered separately, such that the magnetic particles can tag the disease components, and that potentially includes disease components. The magnetic particles can be held in place by the magnetic field and/or magnetic field gradient established by the magnet(s) 108. The magnet(s) 108 can include a group of four magnets with opposite polarities next to each other and the magnetic fields generated by each magnet are shown in FIG. 3. The north side (N) of one of the magnets can be aligned with a south side (S) of another of the magnets, and a magnetic field (or magnetic field gradient) can be established therebetween. Without the magnetic field and/or magnetic field gradient, the magnetic particles (potentially attached to disease components) can be randomly organized with other components in the sample. When exposed to the magnetic field and/or magnetic field gradient, the magnetic particles (potentially attached to disease components) can become organized, aligned, or trapped in any other way within the magnetic field and/or magnetic field gradient while the nonmagnetic components can move without influence through the fluid.

Please note that FIGS. 4 and 5 provide a simplistic, over simplified, qualitative illustration of how the magnet(s) 108 trap the magnetic particles in the microchamber. As shown in FIG. 4, the magnetic particles and binding components (or disease components tagged with magnetic particles and binding components) 302 can be trapped or held by the magnetic field and/or magnetic field gradient, while other components 304 (untagged) of the fluid are non-magnetic and free to move. It should be noted that although all of the other components 304 are illustrated as having the same shape, the other components 304 may be of any conceivable shape and not the same as other of the other components 304. As an example, a fluid (e.g., a buffer) can be forced through the trapping region 104 to remove the other components 304 of the fluid. An example of magnetic particles that can be trapped is shown in FIG. 5. In scenario A represented by 302(A), when the disease component is present, the magnetic particles 402 bound or attached to the binding component 404 can make a complex with the disease component 406 (again the shapes are not important and the different components can have any shape), which is trapped. However, in scenario B represented by 302(B) where there is no disease component, the magnetic particles 402 bound or attached to the binding component 404 can be trapped on their own or be of a small enough size to be washed away.

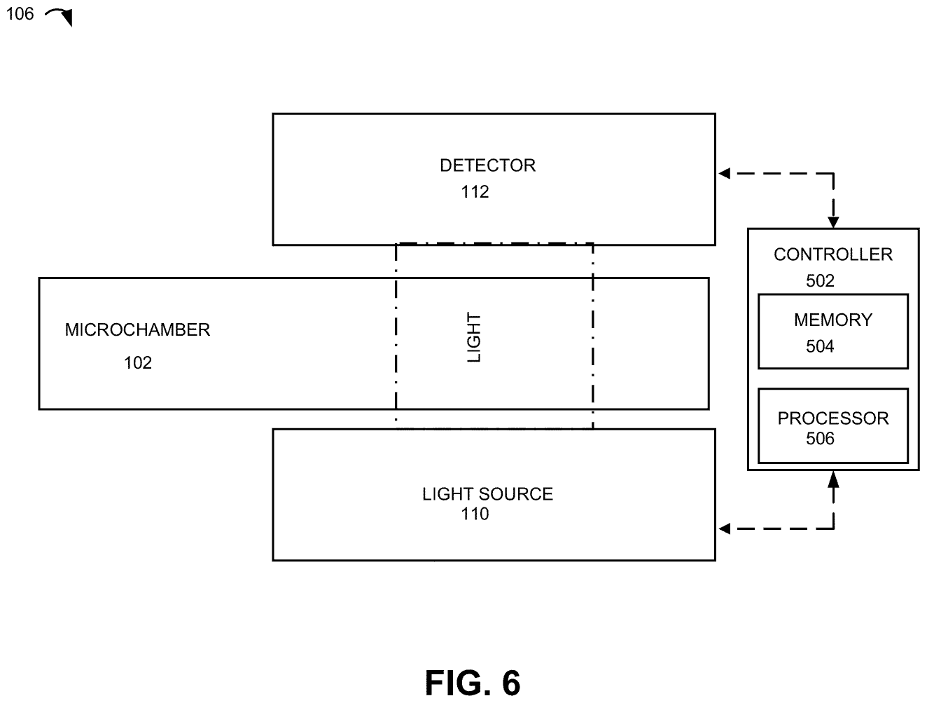

At least one light source device 110 (e.g., one or more lasers, light emitting diodes, light bulbs, etc.) can transmit a beam of light (LIGHT) to at least one detector 112 that can collect light after it passes through the sample to facilitate the detection within the detection region 106 (e.g., the second microcompartment 204), as shown in FIG. 6. The trapping region 104 and the detection region 106 are within the same device. The detection region 106 can be more narrow than the trapping region 104 so that the beam of light illuminates and passes through the entire second microcompartment 204 (e.g., the entire width and/or the entire depth).

The light source 110 and the detector 112 (as well as any additional components that work with the light source 110 and the detector 112) can be collectively referred to as optical instruments.

It will be noted that the at least one light source device 110 and/or the at least one detector 112 can also be associated with one or more controllers (represented as controller 502 with non-transitory memory 504 and a processor 506) or other computing devices, which can be used to operate the at least one light source device 110 and/or the at least one detector 112 in at least a partially automated fashion. For example, the controller or other computing device can interface with one or more components of the at least one light source device 110 and/or the at least one detector 112 to control delivery of light, recording of data, sampling rate of the detector 112, configuration of the diagnostic device, or the like.

The light source 110 can include a laser light source or other type of collimated light source, but the light source 110 can also be a non-collimated light source, like a light bulb or a light emitting diode, with variable wavelengths emitted based on the application (e.g., in instances where fluorescence is used, blue light can be used to cause the fluorophore to fluoresce green light). When the light source 110 is a laser light source, the light from the laser light source can be polarized by a polarizer (e.g. a linear polarizer, a circular polarizer, or the like). As an example, a linear polarizer can create horizontally polarized light. The polarizer can be part of the light source 110 to provide a polarized laser source. A beam splitter can also be part of the light source 110. The beam splitter can aid in power control and/or data collection. Notably, the light produced by the light source can be white light or colored light (of any wavelength).

As an example, the detector 112 can include one or more photodetectors and may be, for example, a camera, a video camera, a fluorescence detector, or the like. Detection by the detector 112 can be controlled by a sampling device (which can be part of controller 502). The sampling device can record detections by the detector 112 according to a sampling frequency. The sampling frequency can differ and be variable based on the application. As an example, the sampling frequency can be sufficient to sample the detector 112 to determine transmission intensities of the light beam (or multiple light beams). The sampling device, as another example, can include a processing unit and can be used to determine the transmission intensities of the light beam (or multiple light beams). Based on the transmission intensities, the sampling device can determine if the disease component exists in the sample.

The device can employ different detection mechanisms. These detection mechanisms are facilitated h the number 102 including the first microcompartment 202 (the trapping region 104) and the second microcompartment 204 (the detection region 106). The magnet(s) 108 can be associated with the first microcornpartment 202 and the light source 110 and the detector 112 can be associated with the second microcompartment 204.

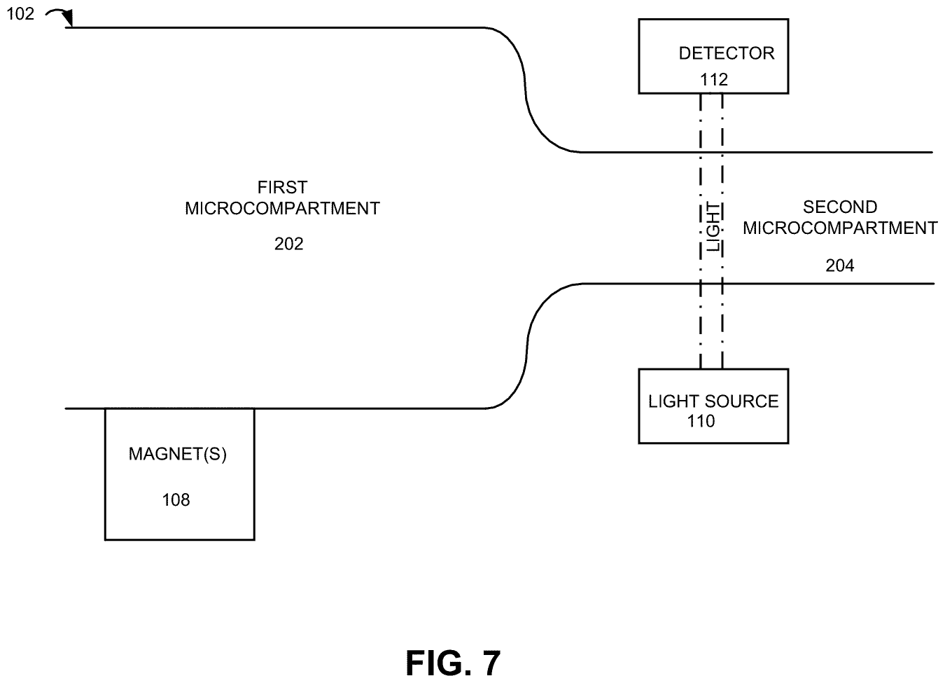

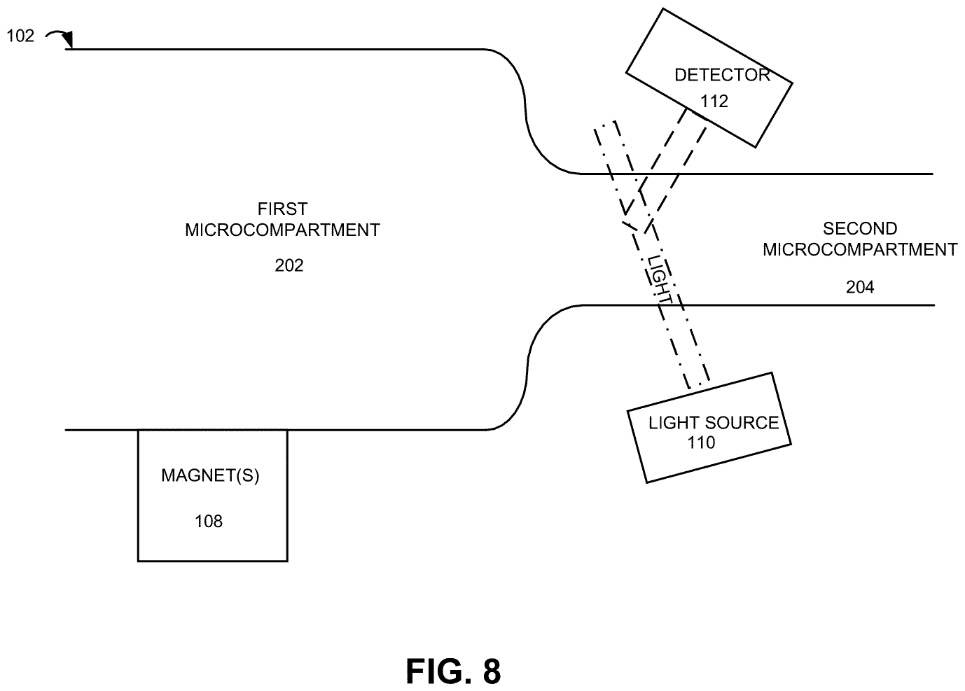

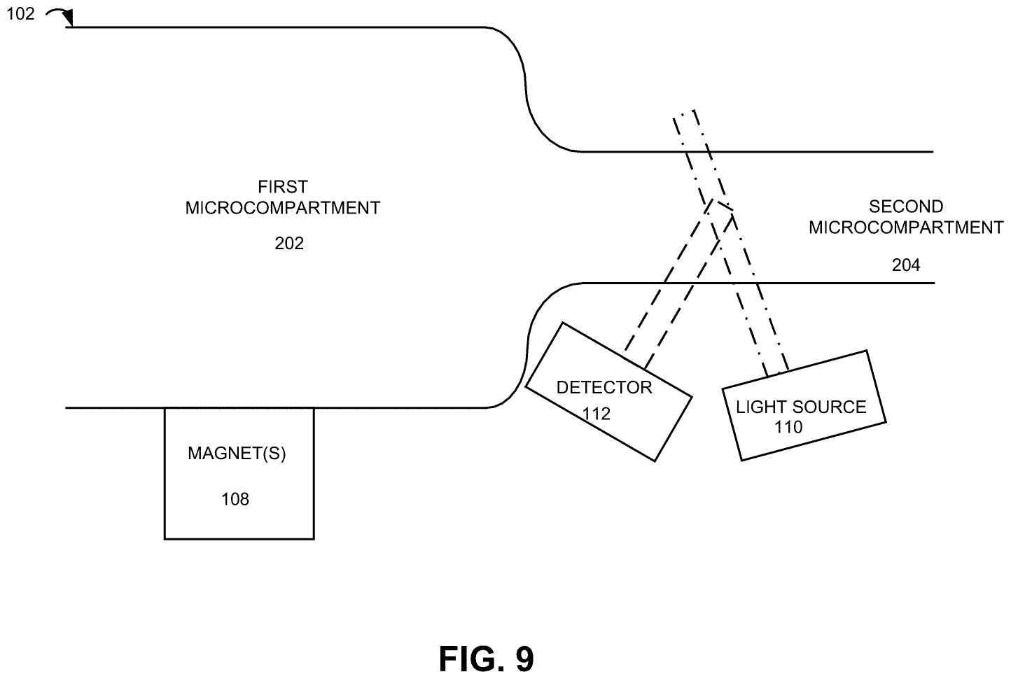

As shown in FIGS. 7-9, the light source 110 can be positioned n different locations relative to the detector 112. One or more filters can be associated with the light source 110 and/or the detector 112.

One detection scheme does not involve fluorescence. A substance can be forced through the channel enabling the detachment of the magnetic particles from the disease components. The disease components, which are no longer magnetic, can be allowed to flow through the second microcompartment 204 (which is smaller than the first rnicrocompartrnent 204). The magnetic particles are still trapped by the magnetic field and/or magnetic field gradient and unable to pass into the second microcompartment 204. The disease component can be detected and counted (either manually or by an automated program) by a detector 112 as the disease component passes through a light beam (or multiple light beams) that passes through the second microcompartment 204. The light beam can be at least partially blocked as the disease components flow past.

Other detection schemes include fluorescence. In one example, the disease components can be tagged with a fluorescent molecule (e.g., FITC). For example, FITC can fluoresce green when exposed to blue light (and the detector 112 can include a green filter). No matter what the fluorescent molecule, the fluorescent molecule can fluoresce at a wavelength that is larger than the wavelength chosen for the initial light source 110 (and the detector 112 can have the respective filter).

The tagged disease component can be imaged and counted in the detection region 106 (however, this does not need to occur in the detection area 106 and can occur in the trapping region 104). As another example, the disease components can be tagged with the fluorescent molecule. As a further example, all components in a solution (e.g., cells) can be tagged generally, but since some may not be bound to the coated magnetic particle (e.g., generic red blood cells) and are washed away, they will not be detected in the subsequent step of detecting circulating tumor cells. A substance can be forced through the channel enabling the detachment of the magnetic particles from the disease components. As another example, the magnet can be turned off and the disease components released. The disease components, which are no longer magnetic, can be allowed to flow through the second microcompartment 202 (which is smaller than the first microcompartment 202), The magnetic particles are still trapped by the magnetic field and/or magnetic field gradient and unable to pass into the second microcompartment 204. The fluorescence of the disease component can be detected and counted (either manually or by an automated program) by a detector as the disease component passes through a light beam (or multiple light beams) through the second microcompartment 204.

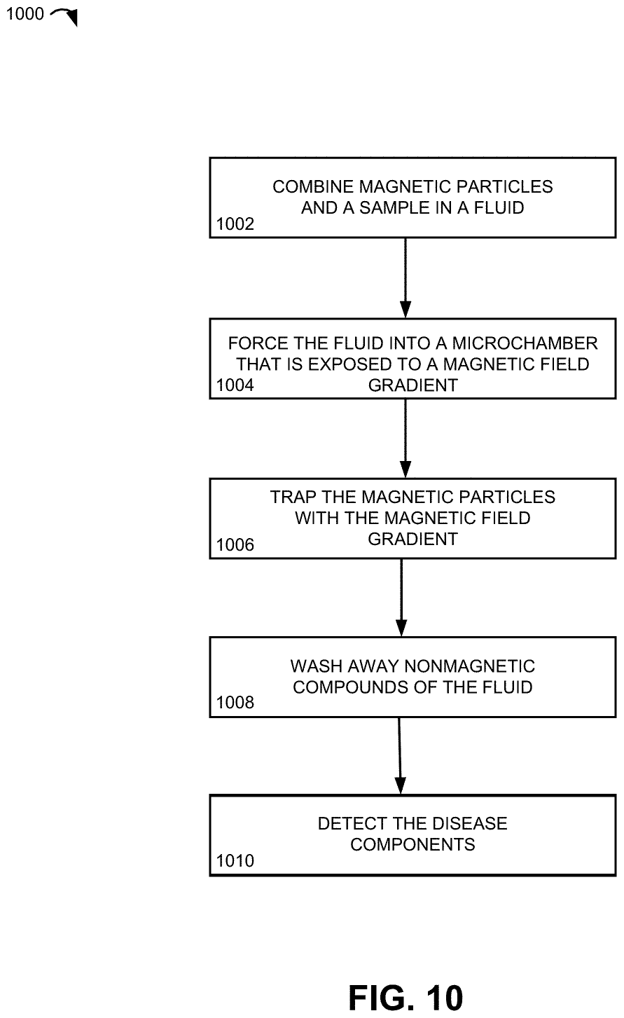

In view of the structural and functional features described above, example methods will be better appreciated with reference to FIGS. 10-12. While, for purposes of simplicity of explanation, the methods of FIGS. 10-12 are shown and described as executing serially, it is to be understood and appreciated that the present invention is not limited by the illustrated order, as some actions could, in other examples, occur in different orders from that shown and described herein or could occur concurrently. It will be appreciated that some or all acts of these methods 1000-1200 can be implemented as machine-readable instructions on a non-transitory computer readable medium.

FIG. 10 illustrates an example method 1000 for performing optical detection of disease components in a fluid using a microchamber and magnetic particles (e.g., as shown in FIG. 1 and the examples in FIGS. 2-9) for trapping and detection of the disease components. At 1002, magnetic particles (or magnetic particles attached or to be attached to a binding component) and a sample can be combined in a fluid (which may be part of the sample, but can also be added to the sample). The magnetic particles are configured to tag disease components within the sample (e.g., through the binding component). For example, the binding component can be one or more antibodies specific for the disease component. The one or more antibodies can each have a plurality of the magnetic particles attached. The tagged antibodies can bind to the disease component, making the disease component magnetic (e.g., tagging the disease component with the magnetic particles).

At 1004, the fluid can be forced into a microchamber that is exposed to a magnetic field and/or a magnetic field gradient. For example, as shown in the trapping portion of FIG. 1. At 1006, the tagged disease component (or the magnetic particles bound to the binding component if no disease component) is trapped by the magnetic field and/or magnetic field gradient (shown in FIG. 3).

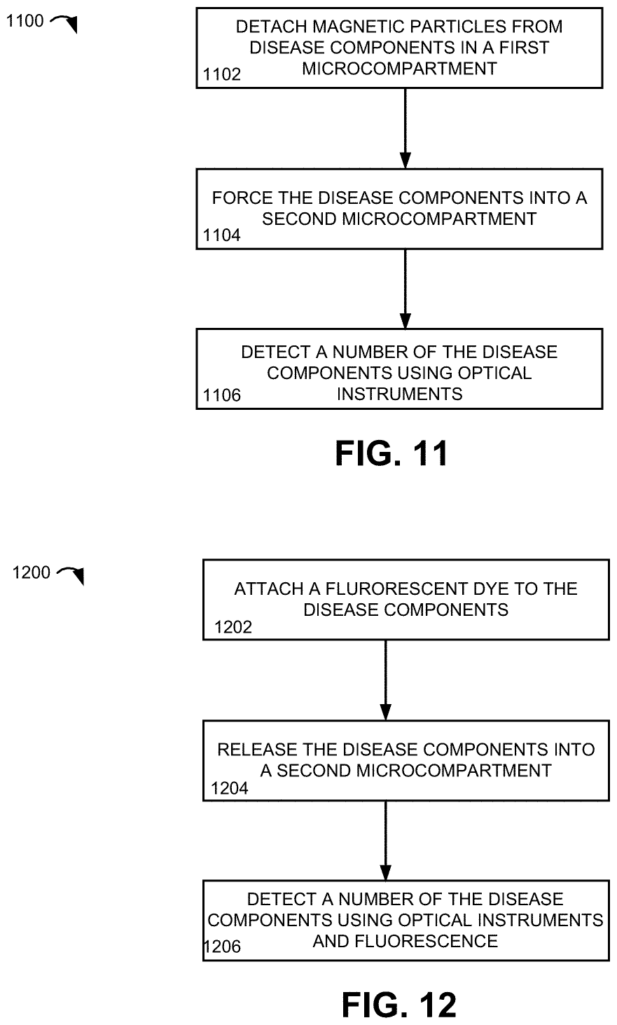

At 1008, nonmagnetic components of the fluid (e.g., element 304) can be washed away (e.g., by another fluid, like a buffer), while the tagged disease components or anything tagged with the magnetic particles (e.g., element 302) remain trapped by the magnetic field and/or magnetic field gradient. At 1010 the disease components within the microchamber (e.g., the detecting portion of FIG. 1) can be detected using optical instruments (e.g., after being forced into the detecting portion by another fluid, such as a buffer). The detecting portion of FIG. 1 can detect disease components as the disease components flow past a light beam or multiple light beams (that in some instances spans the entire depth or width of the detection region) in different ways (e.g., shown in FIGS. 7-9). Two such examples of methods 1100 and 1200 for detecting disease components are shown in FIGS. 11 and 12.

The method 1100 can be undertaken by any of the devices shown in FIGS. 7-9. At 1102, the magnetic particles can be detached from the disease components within a first microcompartment (e.g., in the trapping region of FIG. 1, after the trapping). At 1104, the disease components can be forced into a second microcompartment (e.g., in the detecting region of FIG. 1). At 1106, a number of the disease components can be detected (and, in some instances, counted) using optical instruments (e.g., a light source, the light beam or multiple light beams may be provided by at least one of a laser, a light emitting diode, a light bulb, or the like, and a detector). In some instances, the disease components can be dyed with a fluorescence dye and the fluorescence can be used in the detection.

The method 1200 can also be undertaken by any of the devices shown in FIGS. 7-9. At 1202, fluorescent disease components can be received. The disease components can become fluorescent by a labeled binding component (e.g., a fluorescent antibody) or via a fluorescent agent being added prior to adding the disease components to the chamber (e.g., in the trapping region 104 of FIG. 1). This can occur before or after the trapping. At 1204, the disease components (and potentially the magnetic particles) can be released into a second microcompartment (e.g., in the detecting region of FIG. 1). For example, the magnetic field and/or magnetic field gradient can be removed, releasing both the magnetic particles and the disease components. At 1206, a number of the disease components can be detected (and, in some instances, counted) using optical instruments (e.g., a light source, the light beam or multiple light beams may be provided by at least one of a laser, a light emitting diode, a light bulb, or the like, and a detector) and fluorescence.

As an example, the following experiment provided a demonstration of a method of detecting Borrelia bacteria during early stages of Lyme disease.

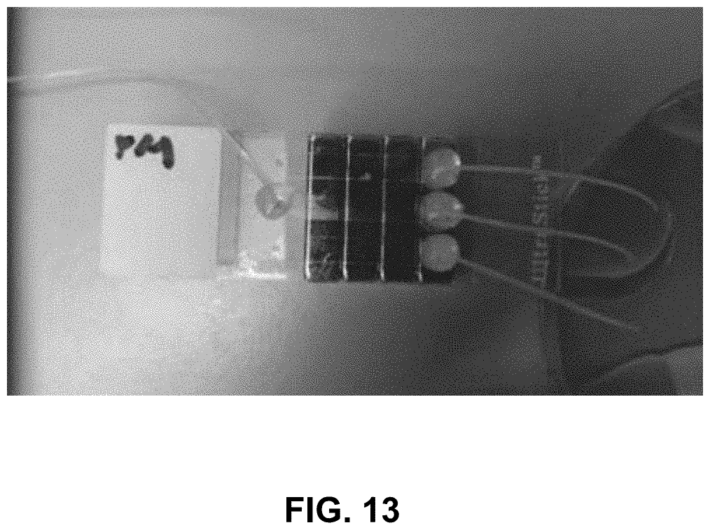

First magnetic microparticles were prepared. The magnetic particles had a one-micron diameter and were functionalized with streptavidin. The magnetic particles were combined with anti-Borrelia burgdorferi antibodies that were conjugated with biotin. Biotin binds to streptavidin, therefore the antibodies coat the surface of the magnetic particles. This results in a suspension of magnetic particles with bound antibodies, as well as free antibodies and possibly free magnetic particles. The free antibodies were then removed from the suspension so as not to take up binding sites on the bacteria that were needed for the magnetic particles or for fluorescent antibodies. The free antibodies were removed from the suspension by forcing the suspension through a microfluidic channel, shown in FIG. 13. The microfluidic device of FIG. 13 had three channels, each 50 microns deep and 0.5 cm wide, with at least one magnet configured on at least one side of the microfluidic device. The magnetic field and/or magnetic field gradient of the at least one magnet traps the magnetic particles. The channel was then flushed with PBS in order to remove any remaining free antibodies. The at least one magnet was removed and the channel was flushed with more PBS in order to force the magnetic particles into a vial. The resultant suspension comprised magnetic particles bound with antibodies and no free antibodies.

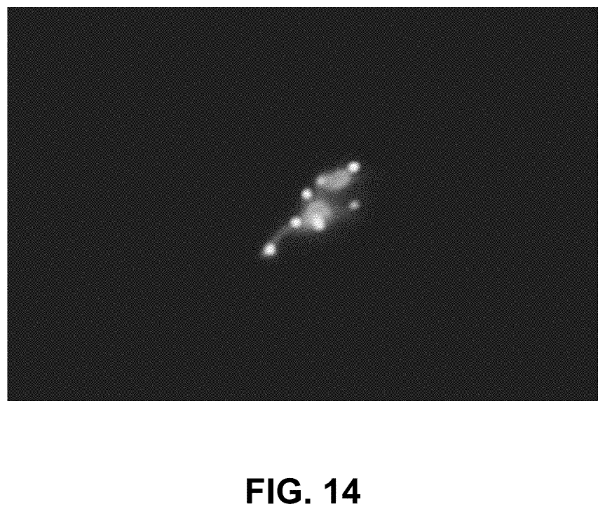

The magnetic particles bound with antibodies were then added to samples containing Borrelia burgdorferi. Anti-Borrelia burgdorferi antibodies conjugated with fluorescent FITC were also added to the samples for imaging. FIG. 14 shows a Borrelia burgdorferi bacteria with attached magnetic particle(s) and FITC antibodies imaged through a fluorescence microscope to show that the magnetic particles prepared as described above indeed bind to the bacteria.

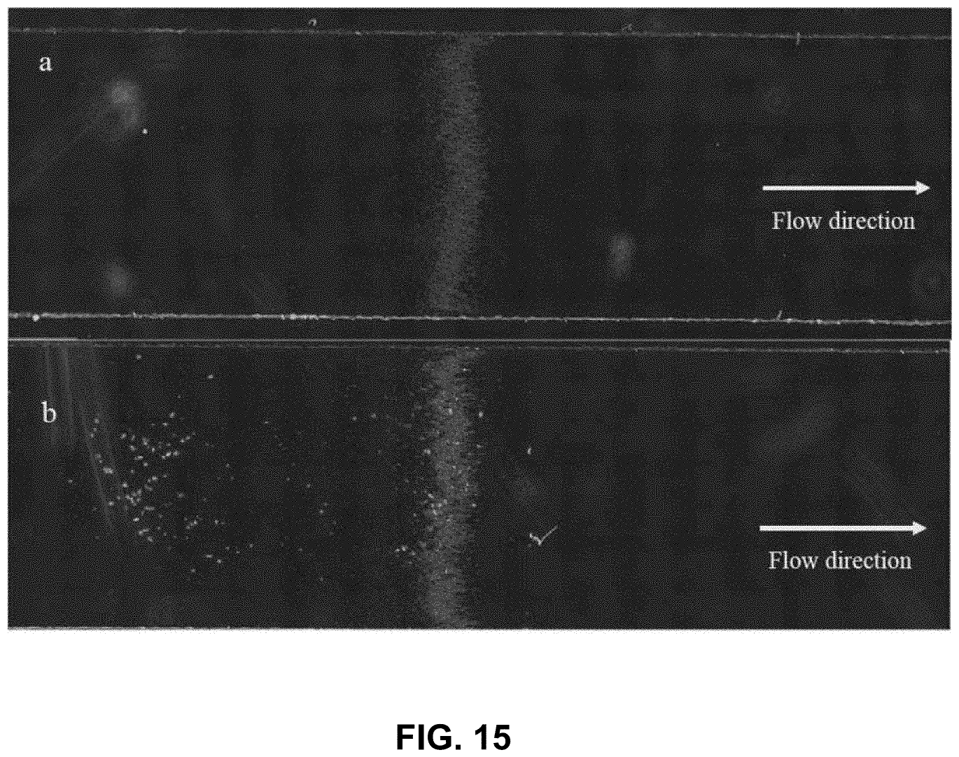

To demonstrate that Lyme bacteria can indeed be trapped in a microfluidic channel, two samples were prepared: a sample without Lyme bacteria to act as a control and a sample containing Lyme bacteria. Magnetic particles with attached antibodies were added to both samples, followed by a fluorescent antibody (FITC). The samples were then added to blood and forced through microfluidic channels, followed by PBS to wash out any material that is nonmagnetic (e.g., red and white blood cells). FIG. 15 shows the results. The top image, image a, shows the sample without Lyme and the bottom image, image b, shows the sample with Lyme. The unattached particles (see grey band near center of channels) are seen to be trapped in both image a and image b, as well as the dots indicating the presence of B. burgdorferi bacteria in image b.

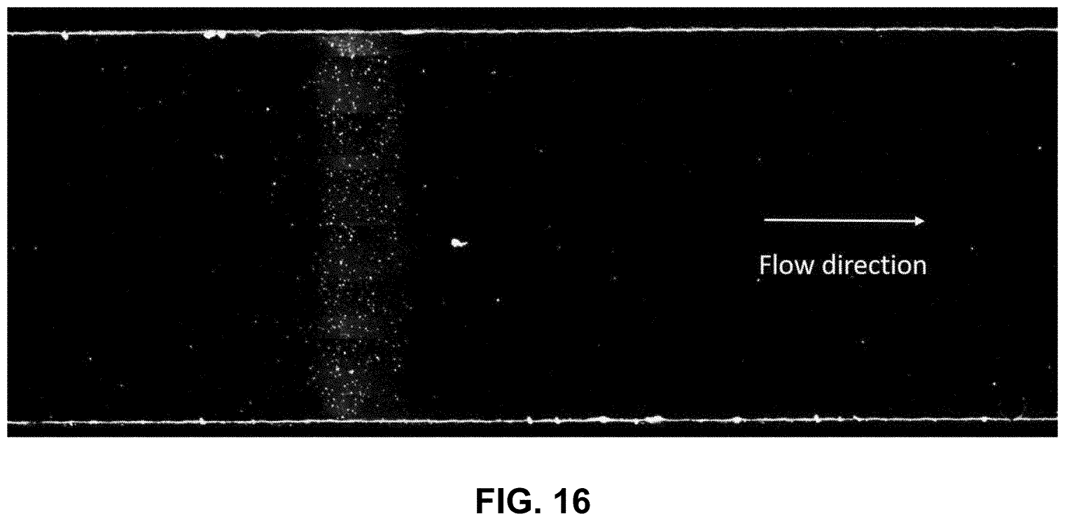

In the previous example, magnetic particles and fluorescent antibodies were first bound to the bacteria and then added to blood. In the next example, bacteria were first added to blood, in order to demonstrate that bacteria already present in the blood can be detected. The following procedure has been followed in the experimental proof of principle. First, magnetic particles with bound antibodies were added to the blood sample. The resultant blood sample was then mechanically mixed to allow magnetic particles to find and become attached to the bacteria. Second, fluorescent antibodies were then added to the blood sample and mechanically mixed to allow the fluorescent antibodies to find and become attached to the bacteria. The blood is then forced through a microfluidic channel positioned on top of permanent magnets. Next, PBS is forced through the channel in order to wash out any nonmagnetic material, such as red and white blood cells, and unbound fluorescent antibodies. Finally, the channel is lifted from the magnets and scanned on a fluorescence microscope. The results are shown in FIG. 16, for which the bacteria are trapped by the magnets (see band of dots). The unbound magnetic particles are not shown so as not to obscure the fluorescing Lyme bacteria.

It should be noted that microfluidic chambers/channels (50 microns deep, 0.5 cm wide, and about 1 inch long were used in the above experiments. However, it should be noted that channels of different dimensions can be used (like any depth less than 1000 microns). For example, the depth need not be microns. Instead, the depth can be any value less than 10 cm.

Other types of disease components can also be tested with the above experiment. As another example, consider cancerous cells such as circulating tumor cells (CTCs), which are indicative of the presence of cancer, and cancer cells obtained from biopsies. CTCs are present in blood in very low concentrations. For example, the concentration of CTCs in blood may be as low as 1 cell/ml of blood and the present system and method can be utilized to detect these very low concentrations. The same method can also be used to separate and detect cancer cells obtained from biopsies.

References to “one embodiment”, “an embodiment”, “some embodiments”, “one example”, “an example”, “some examples” and so on, indicate that the embodiment(s) or example(s) so described may include a particular feature, structure, characteristic, property, element, or limitation, but that not every embodiment or example necessarily includes that particular feature, structure, characteristic, property, element or limitation, Furthermore, repeated use of the phrase “in one embodiment” does not necessarily refer to the same embodiment, though it may.

Where the disclosure or claims recite “a,” “an,” “a first,” or “another” element, or the equivalent thereof, it should be interpreted to include one or more than one such element, neither requiring nor excluding two or more such elements. Furthermore, what have been described above are examples. It is, of course, not possible to describe every conceivable combination of components or methods, but one of ordinary skill in the art will recognize that many further combinations and permutations are possible. Accordingly, the invention is intended to embrace all such alterations, modifications, and variations that fall within the scope of this application, including the appended claims.

What is claimed is:

1. A method comprising:
combining magnetic particles and a sample in a fluid, wherein the magnetic particles are configured to tag disease components within the sample;
forcing the fluid into a microchamber that is exposed to a magnetic field gradient, wherein the tagged disease component is trapped by the magnetic field gradient;
washing away nonmagnetic components of the fluid while the tagged disease components remain trapped by the magnetic field gradient; and
detecting the disease components within the microchamber using optical instruments.

2. The method of claim 1, wherein the detecting further comprises:
detaching the magnetic particles from the disease components in a first microcompartment of the microchamber;
forcing the disease components into a second microcompartment of the microchamber, wherein the second microcompartment of the microchamber has a smaller cross sectional area than the first microcompartment of the microchamber; and
detecting a number of the disease components using the optical instruments as the disease components flow past one or more light beams.

3. The method of claim 2, wherein the one or more light beams span the entire depth or width of the second microcompartment of the microchamber.

4. The method of claim 2, wherein the one or more light beams is provided by at least one of a laser, a light emitting diode, and a light bulb.

5. The method of claim 2, wherein the forcing comprises injecting a fluid into the first microcompartment of the microchamber to detach the magnetic particles from the disease component and/or force the disease component into the second microcompartment of the microchamber.

6. The method of claim 2, further comprising:
attaching a fluorescent dye to the disease components; and
using the one or more light beams to cause the disease components with the fluorescent dye attached to fluoresce.

7. The method of claim 1, wherein the detecting further comprises:
attaching a fluorescent dye to the disease components;
removing the magnetic field gradient from a first microcompartment of the microchamber;
releasing the tagged disease component into a second microcompartment of the microchamber,
wherein the second microcompartment of the microchamber has a smaller cross sectional area than the first microcompartment of the microchamber; and
detecting a number of the disease components as the disease components flow past one or more light beams, wherein the one or more light beams cause the fluorescent dye to fluoresce.

8. The method of claim 1, wherein the detecting comprises counting the disease components.

9. The method of claim 1, wherein the disease component is a bacterium, a virus, a fungus, a parasite, a cell expressing a disease marker, a cell from a tissue biopsy, or a cancer cell.

10. A device comprising:
a microchamber comprising a first microcompartment with a first cross-sectional area and a second microcompartment with a second cross-sectional area, wherein the second cross-sectional area is less than the first cross-sectional area, wherein the first microcompartment is configured to receive a fluid comprising magnetic particles configured to tag disease components;
at least one magnet configured to establish a magnetic field gradient within the first microcompartment of the microchamber, wherein the tagged disease components become trapped by the magnetic field gradient so that nonmagnetic components are washed away; and
a detector configured to detect disease components within the second microcompartment of the microchamber using optical instruments as the disease components flow through the second microcompartment of the microchamber.

11. The device of claim 10, wherein the disease component is a bacterium, a virus, a fungus, a parasite, a cell expressing a disease marker, a cell from a tissue biopsy, or a cancer cell.

12. The device of claim 10, wherein the microchamber has a length of 40 cm or less, a width of 10 cm or less, and a depth of 5 mm or less.

13. The device of claim 10, wherein the microchamber has a length of 10 cm or less, a width of 4 cm or less, and a depth of 1 mm or less.

14. The device of claim 10, wherein the microchamber is a microfluidic channel having a length of 10 cm or less, a width of 1 cm or less, and a depth between 0.01 mm and 0.5 mm.

15. The device of claim 14, wherein the depth varies between the first microcompartment and the second microcompartment.

16. The device of claim 10, wherein at least a portion of the microchamber comprises at least one functionalized surface.

17. The device of claim 10, further comprising a fluid delivery component configured to force a fluid through the microchamber to wash away nonmagnetic components from the first microcompartment.

18. The device of claim 10, wherein the magnetic particles become detached from the disease components in the first microcompartment of the microchamber;
wherein the detector is configured to detect a number of the disease components as the disease components flow past one or more light beams, wherein the one or more light beams span an entire depth or width of the second microcompartment of the microchamber.

19. The device of claim 18, wherein the detector comprises at least one of a laser, a light emitting diode, and a light bulb to provide the one or more light beams.

20. The device of claim 20, wherein a fluorescent dye is attached to the disease components;
and the detector uses the one or more light beams to cause the disease components with the fluorescent dye attached to fluoresce.

21. The device of claim 10, wherein when a fluorescent dye is attached to the disease components, the detector detects a number of the disease components as the disease components flow past one or more light beams, wherein the one or more light beams causes the fluorescent dye to fluoresce.
